(19) United States
(12) Patent Application Publication
(10) Pub. No.: US 2018/0056261 A1
(43) Pub. Date: Mar. 1, 2018

(54) METHOD OF MANUFACTURING COATED BEADS

(71) Applicant: Allergan, Inc., Irvine, CA (US)

(72) Inventors: David J. SCHUESSLER, Santa Ana, CA (US); Erik TORJESEN, Goleta, CA (US); Alberto FLORES-PUJOL, Heredia (CR); Matthew B. MILLER, Hoboken, NJ (US)

(21) Appl. No.: 15/691,637

(22) Filed: Aug. 30, 2017

Related U.S. Application Data
(60) Provisional application No. 62/381,352, filed on Aug. 30, 2016.
(60) Provisional application No. 62/403,465, filed on Oct. 3, 2016.

(51) Int. Cl. B01J 2/00; A61L 31/10; A61L 31/02; A61L 31/06; B01J 2/16

(52) U.S. Cl. CPC B01J 2/006; A61L 31/10; A61L 31/028; A61F 2/12; B01J 2/16; A61L 2420/02; A61L 31/06

(57) ABSTRACT

The present specification discloses methods of making porogen compositions, methods of making polymer-coated beads, and methods of making implantable devices that use polymer-coated beads.

### CROSS-REFERENCE TO RELATED APPLICATIONS

The present application claims priority to U.S. Provisional Application No. 62/381,352, filed Aug. 30, 2016, and U.S. Provisional Application No. 62/403,465, filed Oct. 3, 2016, the entireties of which are incorporated herein by reference.

### BACKGROUND

### Field of the Inventions

The present invention generally relates to methods for manufacturing coated particles or beads, for example, coated particles useful for biomedical and other applications.

### Description of the Related Art

The present disclosure relates to processes commonly known as microencapsulation, fluidized bed coating or Wurster processing. These technologies are used for precision application of coatings or films onto particulate materials, such as powders, crystals, or granules, and other materials. Particulate materials coated with these processes include for example, solid particles with diameters ranging from about 30 μm and up to several centimeters, for example about 100 μm up to about 3 millimeters.

Generally, in particle coating technologies, particles are moved around in the fluidized bed and simultaneously sprayed with a fluid in the form of a solution, suspension or melt. The fluid may be an aqueous or organic solution, for example, a polymeric dispersion. Coating can take place as top spray, tangential spray, or bottom spray or rotor process. Wurster processing generally uses a bottom spray. As the spray contacts the fluidized particles, the aqueous or organic solution of the polymer evaporates and the polymer or other solids it contains forms a thin coating layer or film on each particle. The processes typically involve evaporative removal of an aqueous or organic solvent as the film is deposited. Fluid bed coating processes often include relatively high fluidizing air volume that is used to both circulate the particles and evaporate the solvent.

Wurster processing technology is a common particle coating technology used in many industries, for example, the pharmaceutical industry. Wurster particle coating systems described, for example, in Wurster, U.S. Pat. Nos. 2,648,609, 3,089,824 and 3,253,944, Jones et al. U.S. Pat. Nos. 5,236,503 and 5,437,889, Jensen U.S. Pat. No. 6,685,775, and Bender, et al., U.S. Pat. No. 7,147,717. The entire disclosure of each of these documents is incorporated herein by reference. Wurster processing is used, for example, for coating pharmaceutical products such as beads or tablets. This process is particularly suitable for a controlled release/extended release and delayed/enteric coating of active ingredients layered in the form of a pellet or tablet. Advantageously, by using Wurster processing, a complete sealing of the surface of a particle can be achieved.

Particle coating technologies, including Wurster processing, are useful in other industries as well. For example, films and coatings are applied as a protective layer to particulate materials, for example, to increase shelf life or storage stability of perishable products. Coatings are sometimes applied to particles as a way to increase or improve functionality of particles, for example, as a means to mask odors or tastes, or to release specific active substances.

Coated particles are described in Liu, et al., U.S. Pat. No. 8,685,296, which is incorporated by reference herein in its entirety. Liu et al. discusses porogens comprising a core material and a shell material surrounding the core material, such composite porogens being useful as a sacrificial material for making textured breast implant surfaces. Control of the size and/or shape of the coated particles can be an important factor in this particular application, in that the textured surfaces of biomedical implants made with sacrificial particles provides functionality on a microscopic level. For example, such textured surfaces can be specifically designed and structured to provide some control over cell and tissue ingrowth. It should be appreciated that such biomedical texturing technology could be improved with the availability of highly uniform, well-structured porogens.

For some applications of coated particles, the mechanical properties of the particulate material to be coated are often relevant and sometimes important considerations. For example, the surfaces and even the shapes of the particles themselves can affect quality, consistency and reproducibility of the coating process. In the case of using porogens as a sacrificial material, it is important to achieve coated particles with uniform size, shape, and surfaces, and the coat must have an adequate thickness. In many applications, highly soluble materials such as salts and sugars are chosen as particulate materials for the sacrificial particles. These highly soluble materials are susceptible to chemical and structural damage by solvents used in the coating process, which can affect the resultant size, shape, and surface of the coated particles. Additionally, the susceptibility to damage is increased when a thicker coating is required due to the prolonged exposure of the particulate material. Thus, there is a need for developing methods of coating particles which addresses these problems.

### SUMMARY

In some embodiments of the present disclosure, methods are provided for controlling or enhancing surface properties of particulate materials. In some embodiments, methods are provided for enhancing properties of particulate materials to improve effectiveness and reproducibility of coating using fluidized bed technologies, for example, Wurster processing or other coating processes.

The presently disclosed processes are able to overcome the difficulties described above regarding the use of solvents in coating soluble particulates. The presently disclosed processes achieve highly spherical polymer-coated beads in which the polymer coating has a thickness of at least 10 μm, with the polymer coating showing a smooth surface without significantly damaging the core particulate material.

In some embodiments, a process for making polymer-coated beads is provided comprising providing a particulate material, the particulate material comprising particles; depositing the particulate material into an enclosed zone; fluidizing the particles in the enclosed zone; introducing an aqueous dispersion into the enclosed zone containing the fluidized particles, the aqueous dispersion comprising a polymer component and a solvent component, wherein the solvent component comprises at least 25% by weight of a nonaqueous solvent; allowing the aqueous dispersion to coat the fluidized particles; and allowing or causing the solvent component to evaporate, thereby leaving a polymer coating on the particles, wherein the polymer coating is at least about 10 μm thick.

In some embodiments, a process for manufacturing a soft prosthetic breast implant is provided, the process comprising: forming a flexible shell of silicone elastomer, the silicone elastomer having a thickness; adhering on an exterior of the flexible shell an even distribution of polymer-coated beads produced by a process described herein; curing the flexible shell with the polymer-coated beads adhered thereto; removing the polymer-coated beads thereby forming an open-pored structure on the exterior of the flexible shell, such that the exterior of the flexible shell exhibits an undulating topography, the open-pored structure comprising round cavities defined by impressions of the polymer-coated beads; and processing the flexible shell, such that it forms a closed envelope; wherein the open-pored structure does not extend through an entire thickness of the silicone elastomer.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1A provides a micrograph of polymer-coated beads produced according to Experiment 1 in the Example.

FIG. 1B provides a micrograph of polymer-coated beads produced according to Experiment 3 in the Example.

FIG. 1C provides a micrograph of polymer-coated beads produced according to Experiment 5 in the Example.

FIG. 1D provides a micrograph of polymer-coated beads produced according to Experiment 6 in the Example.

FIG. 1E provides a micrograph of polymer-coated beads produced according to Experiment 7 in the Example.

FIG. 1F provides a micrograph of polymer-coated beads produced according to Experiment 8 in the Example.

FIG. 1G provides a micrograph of polymer-coated beads produced according to Experiment 10 in the Example.

FIG. 1H provides a micrograph of polymer-coated beads produced according to Experiment 11 in the Example.

FIG. 2 provides graph showing the average circularity, average convexity, and the circular equivalent diameter for the polymer-coated beads produced by experiments of the Example.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology Like components are labeled with identical element numbers for ease of understanding.

In some embodiments, a process for making polymer-coated beads is provided comprising providing a particulate material, the particulate material comprising particles; depositing the particulate material into an enclosed zone; fluidizing the particles in the enclosed zone; introducing an aqueous dispersion into the enclosed zone containing the fluidized particles, the aqueous dispersion comprising a polymer component and a solvent component, wherein the solvent component comprises at least 25% by weight of a nonaqueous solvent; allowing the aqueous dispersion to coat the fluidized particles; and allowing or causing the solvent component to evaporate, thereby leaving a polymer coating on the particles, wherein the polymer coating is at least about 10 μm thick.

In some embodiments, the particulate material is a single material. In some embodiments, the particulate material can comprise two or more materials. In some embodiments, the particulate material comprises an inorganic material. In some embodiments, the particulate material is water soluble. In some embodiments, the particulate material comprises an inorganic salt. In some embodiments, the particulate material comprises a sugar.

Useful particulate shapes include, without limitation, roughly spherical, perfectly spherical, ellipsoidal, polyhedronal, triangular, pyramidal, quadrilateral like squares, rectangles, parallelograms, trapezoids, rhombus and kites, and other types of polygonal shapes. In some embodiments, the particulate material has a spherical or nearly spherical shape, which enhances the ability to fluidize the particulate material.

The particulate material can comprise a natural or synthetic, inorganic or organic material. Exemplary materials suitable as a particulate material disclosed herein, include, without limitation, natural and synthetic salts and its derivatives, natural and synthetic sugars and its derivatives, natural and synthetic polysaccharides and its derivatives, natural and synthetic waxes and its derivatives, natural and synthetic metals and its derivatives, natural and synthetic organic solids and its derivatives, natural and synthetic water soluble solids and its derivatives, and/or natural and synthetic polymers and its derivatives, composites thereof, and/or combinations thereof.

The particulate material may be comprised of a single material disclosed herein or a plurality of materials disclosed herein. In some embodiments, a particulate material may comprise, e.g., at least two different materials disclosed herein, at least three different materials disclosed herein, at least four different materials disclosed herein, or at least five different materials disclosed herein. In some embodiments, a particulate material may comprise, e.g., about 1 to about 2 different materials disclosed herein, about 1 to about 3 different materials disclosed herein, about 1 to about 4 different materials disclosed herein, about 1 to about 5 different materials disclosed herein, about 1 to about 6 different materials disclosed herein, about 2 to about 4 different materials disclosed herein, about 2 to about 5 different materials disclosed herein, about 2 to about 6 different materials disclosed herein, about 3 to about 4 different materials disclosed herein, about 3 to about 5 different materials disclosed herein, or about 3 to about 6 different materials disclosed herein.

In some embodiments, the polymer-coated beads comprise a core particulate material having a particle size of, e.g., about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, or about 900 μm. In some embodiments, the polymer-coated beads comprise a coating having a thickness of, e.g., at least 10 μm, at least 20 μm, at least 30 μm, at least 40 μm, at least 50 μm, at least 60 μm, at least 70 μm, at least 80 μm, at least 90 μm, at least 100 μm, at least 200 μm, at least 300 μm, at least 400 μm, at least 500 μm, at least 600 μm, at least 700 μm, at least 800 μm, or at least 900 μm. In some embodiments, the polymer-coated beads comprise a polymer coating having a thickness of, e.g., about 10 μm to about 500 μm, about 10 μm to about 750 μm, about 10 μm to about 1000 μm, about 10 μm to about 2000 μm, about 10 μm to about 3000 μm, about 25 μm to about 500 μm, about 25 μm to about 750 μm, about 25 μm to about 1000 μm, about 25 μm to about 2000 μm, about 25 μm to about 3000 μm, about 50 μm to about 500 μm, about 50 μm to about 750 μm, about 50 μm to about 1000 μm, about 50 μm to about 2000 μm, about 50 μm to about 3000 μm, about 100 μm to about 500 μm, about 100 μm to about 750 μm, about 100 μm to about 1000 μm, about 100 μm to about 2000 μm, or about 100 μm to about 3000 μm.

In some embodiments, the particulate material comprises an inorganic material. In some embodiments, the particulate material comprises an organic material. In some embodiments, the particulate material comprises a salt and/or its derivatives, a sugar and/or its derivatives, a polysaccharide and/or its derivatives, a wax and/or its derivatives, a metal and/or its derivatives, a water soluble solid and/or its derivatives, or a polymer and/or its derivatives.

In some embodiments, the particulate material comprises an inorganic salt particle. The inorganic salt particles can comprise, for example, any ionic compound that is naturally crystalline, such as ordinary table salt, i.e., sodium chloride (NaCl). The particles may comprise, alternatively, potassium chloride or calcium carbonate, for example, or combinations thereof. Other non-limiting examples of suitable salts include lithium chloride, magnesium chloride, calcium chloride, ammonium chloride, sodium iodide, potassium iodide, lithium iodide, magnesium iodide, calcium iodide, ammonium iodide, sodium bromide, potassium bromide, lithium bromide, magnesium bromide, calcium bromide, ammonium bromide, sodium carbonate, potassium carbonate, lithium carbonate, magnesium carbonate, ammonium carbonate, sodium bicarbonate, potassium bicarbonate, lithium bicarbonate, ammonium bicarbonate, sodium nitrate, potassium nitrate, lithium nitrate, magnesium nitrate, calcium nitrate, ammonium nitrate, sodium acetate, potassium acetate, lithium acetate, magnesium acetate, calcium acetate, ammonium acetate, sodium phosphate, potassium phosphate, lithium phosphate, magnesium phosphate, calcium phosphate, ammonium phosphate, sodium sulfate, potassium sulfate, lithium sulfate, magnesium sulfate, calcium sulfate, or ammonium sulfate, and combinations thereof. A person of skill in the art will recognize the suitability several other salt materials not disclosed herein. For use in the manufacture of medical implants, the salt particles are preferably biocompatible and safe to use in human beings.

In some embodiments, the inorganic salt particles are rounded, spherical, or nearly spherical salt particles. Methods for producing rounded salt particles have been disclosed in Schuessler et al., U.S. application Ser. No. 15/607,338, the entirety of which is incorporated herein by reference.

A particle's “sphericity” or “mean circularity” can be determined using the surface area of a sphere having the same volume as the particle, divided by the actual surface area of the particle. For example, a sphericity of 1.00 represents a perfect sphere. Particle sphericities can be determined using a Malvern (Westborough, Mass., USA) Morphologi G3 ID instrument. Other methods for measuring the particle's sphericity may be utilized and will be readily apparent to a person of skill in the art.

In some embodiments, the salt particles have a sphericity of greater than 0.750. For example, in some embodiments, the salt particles have a sphericity of greater than 0.800, greater than 0.850, greater than 0.900, greater than 0.930, or greater than 0.950. In some embodiments, the salt particles have a sphericity of about 0.950, which provides excellent physical properties that are superior to angular or cubic shaped particles and useful in a variety of industries, such as the textile, dairy, food, fertilizer, paper, pharmaceutical, and medical device industries.

The rounded, spherical or nearly spherical salt particles may have a size, for example, in the range of about 100 μm to about 1200 μm, about 200 μm to about 1000 μm, about 400 μm to about 800 μm, or about 500 μm to about 700 μm. The method may provide, for example, such salt particles having a size or diameter of about 100 μm, about 200 μm, about 300 μm, about 400 μm, about 500 μm, about 600 μm, about 700 μm, about 800 μm, about 900 μm, about 1000 μm, about 1100 μm, or about 1200 μm, depending on the desired use of the product.

In some embodiments, the particulate material comprises a natural or synthetic sugar. In some embodiments, the particulate material comprises a monomeric sugar compound, i.e., a monosaccharide. In some embodiments, the particulate material comprises a polysaccharide of up to 10 monosaccharide units, e.g., a disaccharide, a trisaccharide, and an oligosaccharide comprising four to ten monosaccharide units. Monosaccharides are polyhydroxy aldehydes or polyhydroxy ketones with three or more carbon atoms, including aldoses, dialdoses, aldoketoses, ketoses and diketoses, as well as cyclic forms, deoxy sugars and amino sugars, and their derivatives, provided that the parent monosaccharide has a (potential) carbonyl group. Oligosaccharides are compounds in which at least two monosaccharide units are joined by glycosidic linkages. According to the number of units, they are called disaccharides, trisaccharides, tetrasaccharides, pentasaccharides, hexoaccharides, heptoaccharides, octoaccharides, nonoaccharides, decoaccharides, etc. An oligosaccharide can be unbranched, branched or cyclic. Non-limiting examples of sugars include, monosacchrides, such as, e.g., trioses, like glyceraldehyde and dihydroxyacetone; tetroses, like erythrose, threose and erythrulose; pentoses, like arabinose, lyxose, ribose, xylose, ribulose, xylulose; hexoses, like allose, altrose, galactose, glucose, gulose, idose, mannose, talose, fructose, psicose, sorbose, tagatose, fucose, rhamnose; heptoses, like sedoheptulose and mannoheptulose; octooses, like octulose and 2-keto-3-deoxy-manno-octonate; nonoses like sialose; and decose; and oligosaccharides, such as, e.g., disaccharides, like sucrose, lactose, maltose, trehalose, cellobiose, gentiobiose, kojibiose, laminaribiose, mannobiose, melibiose, nigerose, rutinose, and xylobiose; trisaccharides like raffinose, acarbose, maltotriose, and melezitose and/or mixtures thereof. Sugars also include sugar substitutes like acesulfame potassium, alitame, aspartame, acesulfame, cyclamate, dulcin, glucin, neohesperidin dihydrochalcone, neotame, saccharin, and sucralose.

In some embodiments, the particulate material comprises rounded, spherical, or nearly spherical sugar particles. In some embodiments, the spherical sugar particles comprise sucrose. In some embodiments, the spherical particles comprise starch. In some embodiments, the spherical particles comprise a combination of sucrose and starch. Further, in some embodiments, the spherical particles can comprise about 1% by weight to about 40% by weight, about 10% by weight to about 30% by weight, or about 15% by weight to about 25% by weight of starch. In some embodiments, the spherical particles comprise about 20% by weight of starch. Spherical sugar particles can be acquired commercially, for example, the Suglet® from Colorcon, Inc. (Irvine, Calif., USA).

The particulate material is deposited or placed into an enclosed zone in preparation for coating the particles. In some embodiments, the enclosed zone comprises part of an apparatus for fluid bed coating or Wurster processing. In some embodiments, the enclosed zone comprises part of an apparatus for microencapsulation or rotor processing.

After the particulate material is placed or deposited into an enclosed zone, the particles are fluidized. As used herein, the term “fluidization” refers to the process of converting a particulate material from a static solid-like state to a dynamic fluid-like state. When fluidized, a bed of solid particles will behave as a fluid, like a liquid or gas. The fluid properties allow the particles to conform to the volume of the enclosed zone and to be transported through enclosed spaces in a similar manner as liquids and gases.

The polymer coatings of the present disclosure are applied to the particulate material through an aqueous dispersion comprising a polymer component and a solvent component. The solvent component will comprise at least 25% by weight of a nonaqueous solvent.

The polymer component can comprise any suitable polymer for coating the particulate material. The polymeric component can comprise natural and synthetic polymers and derivatives thereof. The polymer component can comprise a single polymeric material or a plurality of polymeric materials. In some embodiments, the polymer component can comprise a natural or synthetic elastomer.

A natural or synthetic elastomer or elastic polymer refers to an amorphous polymer that exists above its glass transition temperature at ambient temperatures, thereby conferring the property of viscoelasticity so that considerable segmental motion is possible, and includes, without limitation, carbon-based elastomers, silicon-based elastomers, thermoset elastomers, and thermoplastic elastomers. As used herein, the term “ambient temperature” refers to a temperature of about 18° C. to about 22° C. Elastomers, ether naturally occurring or synthetically made, comprise monomers usually made of carbon, hydrogen, oxygen, and/or silicon which are linked together to form long polymer chains. Elastomers are typically covalently cross-linked to one another, although non-covalently cross-linked elastomers are known. Elastomers may be homopolymers or copolymers, degradable, substantially non-degradable, or non-degradable. Copolymers may be random copolymers, blocked copolymers, graft copolymers, and/or mixtures thereof. Unlike other polymers classes, elastomers can be stretched many times its original length without breaking by reconfiguring themselves to distribute an applied stress, and the cross-linkages ensure that the elastomers will return to their original configuration when the stress is removed. Elastomers can be a non-medical grade elastomer or a medical grade elastomer. Medical grade elastomers are typically divided into three categories: non-implantable, short term implantable and long-term implantable. Exemplary substantially non-degradable and/or non-degradable, biocompatible, elastomers include, without limitation, bromo isobutylene isoprene (BUR), polybutadiene (BR), chloro isobutylene isoprene (CIIR), polychloroprene (CR), chlorosulphonated polyethylene (CSM), ethylene propylene (EP), ethylene propylene diene monomer (EPDM), fluorinated hydrocarbon (FKM), fluoro silicone (FVQM), hydrogenated nitrile butadiene (HNBR), polyisoprene (IR), isobutylene isoprene butyl (IIR), methyl vinyl silicone (MVQ), acrylonitrile butadiene (NBR), polyurethane (PU), styrene butadiene (SBR), styrene ethylene/butylene styrene (SEBS), polydimethylsiloxane (PDMS), polysiloxane (SI), and acrylonitrile butadiene carboxy monomer (XNBR).

A natural or synthetic polymer and its derivatives, refer to natural and synthetic macromolecules composed of repeating structural units typically connected by covalent chemical bonds. A polymer includes natural or synthetic hydrophilic polymers, natural or synthetic hydrophobic polymers, natural or synthetic amphiphilic polymers, degradable polymers, partially degradable polymers, non-degradable polymers, and combinations thereof. Polymers may be homopolymers or copolymers. Copolymers may be random copolymers, blocked copolymers, graft copolymers, and/or mixtures thereof. Non-limiting examples of polymers include poly(alkylene oxide), poly(acrylamide), poly(acrylic acid), poly(acrylamide-co-arylic acid), poly(acrylamide-co-diallyldimethylammonium chloride), poly(acrylonitrile), poly(allylamine), poly(amide), poly(anhydride), poly(butylene), poly(ε-caprolactone), poly(carbonate), poly(ester), poly(etheretherketone), poly(ethersulphone), poly(ethylene), poly(ethylene alcohol), poly(ethylenimine), poly(ethylene glycol), poly(ethylene oxide), poly(glycolide) ((like poly(glycolic acid)), poly(hydroxy butyrate), poly(hydroxyethylmethacrylate), poly(hydroxypropylmethacrylate), poly(hydroxystrene), poly(imide), poly(lactide), poly(L-lactic acid), poly(D,L-lactic acid), poly(lactide-co-glycolide), poly(lysine), poly(methacrylate), poly(methacrylic acid), poly(methylmethacrylate), poly(orthoester), poly(phenylene oxide), poly(phosphazene), poly(phosphoester), poly(propylene fumarate), poly(propylene), poly(propylene glycol), poly(propylene oxide), poly(styrene), poly(sulfone), poly(tetrafluoroethylene), poly(vinyl acetate), poly(vinyl alcohol), poly(vinyl chloride), poly(vinylidene fluoride), poly(vinyl pyrrolidone), poly(urethane), collagen, gelatin, any copolymer thereof (like poly(ethylene oxide) poly(propylene oxide) copolymers (poloxamers), poly(vinyl alcohol-co-ethylene), poly(styrene-co-allyl alcohol, and poly(ethylene)-block-poly(ethylene glycol), and/or any mixtures thereof. In some embodiment, the polymer component comprises polyethylene glycol. In some embodiments, the polymer coating comprises polyethylene glycol.

The polymer component and/or polymer coating may be comprised of a single material disclosed herein or a plurality of materials disclosed herein. In some embodiments, the polymer component and/or polymer coating may comprise, e.g., at least two different materials disclosed herein, at least three different materials disclosed herein, at least four different materials disclosed herein, or at least five different materials disclosed herein. In some embodiments, the polymer component and/or polymer coating may comprise, e.g., about 1 to about 2 different materials disclosed herein, about 1 to about 3 different materials disclosed herein, about 1 to about 4 different materials disclosed herein, about 1 to about 5 different materials disclosed herein, about 1 to about 6 different materials disclosed herein, about 2 to about 4 different materials disclosed herein, about 2 to about 5 different materials disclosed herein, about 2 to about 6 different materials disclosed herein, about 3 to about 4 different materials disclosed herein, about 3 to about 5 different materials disclosed herein, or about 3 to about 6 different materials disclosed herein.

The polymer coating has a thickness sufficient to allow formation of a porogen scaffold. As a result, the polymer coating can be of any thickness, with the proviso that the amount of polymer is sufficient to create a porogen scaffold useful for its intended purpose. The thickness of the polymer coating is measured from the inner surface of the coating that is adjacent of the core particulate material to the outer surface of the polymer coating.

In some embodiments, the polymer-coated beads comprise a polymer coating having a thickness sufficient to allow formation of a porogen scaffold. In some embodiments, the polymer-coated beads comprise a polymer coating having a thickness of, e.g., about 1 μm, about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, or about 50 μm. In some embodiments, the polymer-coated beads comprise a polymer coating having a thickness of, e.g., at least 1 μm, at least 2 μm, at least 3 μm, at least 4 μm, at least 5 μm, at least 6 μm, at least 7 μm, at least 8 μm, at least 9 μm, at least 10 μm, at least 15 μm, at least 20 μm, at least 25 μm, at least 30 μm, at least 35 μm, at least 40 μm, at least 45 μm, or at least 50 μm. In some embodiments, the polymer-coated beads comprise a polymer coating having a thickness of, e.g., about 5 μm to about 50 μm, about 5 μm to about 75 μm, about 5 μm to about 100 μm, about 5 μm to about 200 μm, about 5 μm to about 300 μm, about 10 μm to about 50 μm, about 10 μm to about 75 μm, about 10 μm to about 100 μm, about 10 μm to about 200 μm, about 10 μm to about 300 μm, about 15 μm to about 50 μm, about 15 μm to about 75 μm, about 15 μm to about 100 μm, about 15 μm to about 200 μm, about 15 μm to about 300 μm, about 25 μm to about 50 μm, about 25 μm to about 75 μm, about 25 μm to about 100 μm, about 25 μm to about 200 μm, about 25 μm to about 300 μm, about 35 μm to about 50 μm, about 35 μm to about 75 μm, about 35 μm to about 100 μm, about 35 μm to about 200 μm, or about 35 μm to about 300 μm.

The solvent component can comprise water and a nonaqueous solvent. In some embodiments, the nonaqueous solvent is an organic solvent. In some embodiments, the nonaqueous solvent is miscible with water. In some embodiments, the nonaqueous solvent is selected from a C1 to C4 alcohol, acetone, methyl ethylene ketone, dimethylformamide, ethylene glycol, dichloromethane, chloroform, and combinations thereof. In some embodiments, the nonaqueous solvent comprises a solvent selected from ethanol, methanol, and combinations thereof. In some embodiments, the nonaqueous solvent comprises ethanol.

The solvent component comprises at least about 25% by weight of a nonaqueous solvent. In some embodiments, the solvent component comprises between about 25% and about 90% by weight of a nonaqueous solvent. In some embodiments, the solvent component comprises between about 30% and about 80% by weight of a nonaqueous solvent. In some embodiments, the solvent component comprises between about 30% and about 80% by weight of a nonaqueous solvent. In some embodiments, the solvent component comprises between about 40% and about 70% by weight of a nonaqueous solvent. In some embodiments, the solvent component comprises between about 40% and about 60% by weight of a nonaqueous solvent. In some embodiments, the solvent component comprises between about 45% and about 55% by weight of a nonaqueous solvent. In some embodiments, the solvent component comprises between about 50% and about 70% by weight of a nonaqueous solvent. In some embodiments, the solvent component comprises between about 50% and about 60% by weight of a nonaqueous solvent. In some embodiments, the solvent component comprises between about 50% and about 55% by weight of a nonaqueous solvent. In some embodiments, the solvent component comprises between about 60% and about 90% by weight of a nonaqueous solvent. In some embodiments, the solvent component comprises between about 60% and about 80% by weight of a nonaqueous solvent. In some embodiments, the solvent component comprises between about 70% and about 80% by weight of a nonaqueous solvent. In some embodiments, the solvent component is about 50% by weight ethanol and 50% by weight water. In some embodiments, the solvent component is about 75% by weight ethanol and 25% by weight water.

The polymer component can be present in the aqueous dispersion in any suitable amount. In some embodiments, the aqueous dispersion comprises about 1% by weight to about 40% by weight of the polymer component. In some embodiments, the aqueous dispersion comprises about 5% by weight to about 35% by weight of the polymer component. In some embodiments, the aqueous dispersion comprises about 10% by weight to about 30% by weight of the polymer component. In some embodiments, the aqueous dispersion comprises about 15% by weight to about 25% by weight of the polymer component. In some embodiments, the aqueous dispersion comprises about 20% by weight to about 30% by weight of the polymer component. In some embodiments, the aqueous dispersion comprises about 22% by weight to about 28% by weight of the polymer component. In some embodiments, the aqueous dispersion comprises about 24% by weight to about 26% by weight of the polymer component. In some embodiments, the aqueous dispersion comprises about 15% by weight, about 16% by weight, about 17% by weight, about 18% by weight, about 19% by weight, about 20% by weight, about 21% by weight, about 22% by weight, about 23% by weight, about 24% by weight, about 25% by weight, about 26% by weight, about 27% by weight, about 28% by weight, about 29% by weight, about 30% by weight, about 31% by weight, about 31% by weight, about 32% by weight, about 33% by weight, about 34% by weight, or about 35% by weight of the polymer component. In some embodiments, the aqueous dispersion comprises about 25% by weight of the polymer component.

Coating a particle with polymer can be accomplished by any suitable means, including, without limitation, mechanical application such as, e.g., dipping, spraying, filtration, knifing, curtaining, brushing, or vapor deposition; physical adsorption application; thermal application; fluidization application; adhering application; chemical bonding application; self-assembling application; molecular entrapment application, and/or any combination thereof. The polymer coating is applied to the particle of core material in such a manner as to coat the particle with the desired thickness of polymer. Removal of excess polymer material can be accomplished by any suitable means, including, without limitation, gravity-based filtering or sieving, vacuum-based filtering or sieving, blowing, and/or any combination thereof.

In some embodiments, the aqueous dispersion is introduced into the enclosed zone in any suitable manner for coating the particle. In some embodiments, the aqueous dispersion is introduced into the enclosed zone by spraying, ultrasonic spraying, injecting, misting, nebulizing, or aerosolizing the aqueous dispersion. In some embodiments, the step of introducing an aqueous dispersion comprises spraying the aqueous dispersion into the enclosed zone. In some embodiments, the aqueous dispersion is sprayed into the enclosed zone as a top spray, a tangential spray, a bottom spray, or combinations thereof. In some embodiments, the aqueous dispersion is sprayed into the enclosed zone as a bottom spray.

In some embodiments, the aqueous dispersion is sprayed into the enclosed zone at a rate of between about 25 grams/min and about 65 grams/min. In some embodiments, the aqueous dispersion is sprayed into the enclosed zone at a rate of between about 25 grams/min and about 45 grams/min. In some embodiments, the aqueous dispersion is sprayed into the enclosed zone at a rate of between about 45 grams/min and about 65 grams/min. In some embodiments, the aqueous dispersion is sprayed into the enclosed zone at a rate of between about 25 grams/min and about 35 grams/min. In some embodiments, the aqueous dispersion is sprayed into the enclosed zone at a rate of between about 35 grams/min and about 45 grams/min. In some embodiments, the aqueous dispersion is sprayed into the enclosed zone at a rate of between about 55 grams/min and about 65 grams/min. In some embodiments, the aqueous dispersion is sprayed into the enclosed zone at a rate of about 25 grams/min, about 26 grams/min, about 27 grams/min, about 28 grams/min, 29 grams/min, about 30 grams/min, about 31 grams/min, about 32 grams/min, about 33 grams/min, about 34 grams/min, about 35 grams/min, about 36 grams/min, 37 grams/min, about 38 grams/min, about 39 grams/min, about 40 grams/min, about 41 grams/min, about 42 grams/min, about 43 grams/min, about 44 grams/min, 45 grams/min, about 46 grams/min, about 47 grams/min, about 48 grams/min, about 49 grams/min, about 50 grams/min, about 51 grams/min, about 52 grams/min, 53 grams/min, about 54 grams/min, about 55 grams/min, about 56 grams/min, about 57 grams/min, about 58 grams/min, about 59 grams/min, about 60 grams/min, 61 grams/min, about 62 grams/min, about 63 grams/min, about 64 grams/min, or about 65 grams/min. In some embodiments, the aqueous dispersion is sprayed into the enclosed zone at a rate of about 25 grams/min. In some embodiments, the aqueous dispersion is sprayed into the enclosed zone at a rate of about 45 grams/min. In some embodiments, the aqueous dispersion is sprayed into the enclosed zone at a rate of about 65 grams/min.

In some embodiments, the temperature of the enclosed zone is controlled while the aqueous dispersion is being introduced or sprayed therein. In some embodiments, the temperature in the enclosed zone during the spraying is between about 25° C. and about 40° C. In some embodiments, the temperature in the enclosed zone during the spraying is between about 25° C. and about 35° C. In some embodiments, the temperature in the enclosed zone during the spraying is between about 25° C. and about 30° C. In some embodiments, the temperature in the enclosed zone during the spraying is between about 30° C. and about 40° C. In some embodiments, the temperature in the enclosed zone during the spraying is between about 35° C. and about 40° C. In some embodiments, the temperature in the enclosed zone during the spraying is about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

After the aqueous dispersion is introduced into the enclosed zone, it will contact the fluidized particles contained therein. The polymer coating forms as the solvent component of the aqueous dispersion become devolitized. As used herein, the term “devolitalizing” or “devolitalization” refers to a process that removes volatile components (e.g., solvent component) from a substance base (e.g., aqueous dispersion) or a particle coated with the aqueous dispersion and/or the forming polymer layer. Devolitalization of a substance base and/or a particle coated with the aqueous dispersion and/or the forming polymer layer can be accomplished by any suitable means that substantially all the volatile components are removed from the resultant polymer-coated beads. Non-limiting examples of devolitalizing procedures include evaporation, freeze-drying, sublimation, extraction, and/or any combination thereof. The application of these techniques will be readily apparent to a person of ordinary skill in the art. In some embodiments, the aqueous dispersion is devolitized by allowing the solvent component to evaporate. In some embodiments, the aqueous dispersion is devolitized by evaporating the solvent component at an increased temperature such as the temperatures listed above that are maintained while spraying the aqueous dispersion.

In some embodiments, the process for making polymer-coated beads will have a duration of about 1 hour to about 10 hours. In some embodiments, the process for making polymer-coated beads will have a duration of about 2 hour to about 9 hours. In some embodiments, the process for making polymer-coated beads will have a duration of about 3 hour to about 8 hours. In some embodiments, the process for making polymer-coated beads will have a duration of about 4 hour to about 7 hours. In some embodiments, the process for making polymer-coated beads will have a duration of about 5 hour to about 8 hours. In some embodiments, the process for making polymer-coated beads will have a duration of about 6 hour to about 8 hours. In some embodiments, the process for making polymer-coated beads will have a duration of at least about 1 hour. In some embodiments, the process for making polymer-coated beads will have a duration of at least about 2 hours. In some embodiments, the process for making polymer-coated beads will have a duration of at least about 3 hours. In some embodiments, the process for making polymer-coated beads will have a duration of at least about 4 hours. In some embodiments, the process for making polymer-coated beads will have a duration of at least about 5 hours. In some embodiments, the process for making polymer-coated beads will have a duration of at least about 6 hours. In some embodiments, the process for making polymer-coated beads will have a duration of at least about 7 hours. In some embodiments, the process for making polymer-coated beads will have a duration of at least about 8 hours. In some embodiments, the process for making polymer-coated beads will have a duration of at least about 9 hours.

In some embodiments, the polymer-coated beads have a sphericity of greater than 0.750. For example, in some embodiments, the polymer-coated beads have a sphericity of greater than 0.800, greater than 0.850, greater than 0.900, greater than 0.930, or greater than 0.950. In some embodiments, the polymer-coated beads have a sphericity of about 0.950, which provides excellent physical properties that are superior to angular or cubic shaped particles and useful in a variety of industries, such as the textile, dairy, food, fertilizer, paper, pharmaceutical, and medical device industries.

In some embodiments, rounded, spherical or nearly spherical polymer-coated beads may have a size, for example, in the range of about 100 μm to about 1200 μm, about 200 μm to about 1000 μm, about 400 μm to about 800 μm, or about 500 μm to about 700 μm. The method may provide, for example, such polymer-coated beads having a size or diameter of about 100 μm, about 200 μm, about 300 μm, about 400 μm, about 500 μm, about 600 μm, about 700 μm, about 800 μm, about 900 μm, about 1000 μm, about 1100 μm, or about 1200 μm, depending on the desired use of the product.

### Medical Implants

In some embodiments, the methods disclosed herein can provide polymer-coated beads useful for texturing medical implants, for example, breast implants. The polymer-coated beads can have a size of about 400 μm to about 600 μm, for example, about 500 μm. Methods for applying porogens or sacrificial particles such as polymer-coated beads in the manufacture of textured medical implants are described in detail in U.S. Pat. No. 8,313,527, the entirety of which is incorporated herein by reference. A person of skill in the art would understand how to apply the polymer-coated beads formed from the processes described herein as a sacrificial material in the processes for manufacturing these and other medical implants in which a surface texture or other attribute may be desirable.

For example, in some embodiments, a breast implant having an elastomeric silicone shell can be processed to create a desired surface texture by using polymer-coated beads as a sacrificial material. The polymer-coated beads can be those produced by the present methods.

The processes for forming the breast implant generally comprise the steps of forming a flexible shell, adhering on the exterior of the flexible shell a distribution of polymer-coated beads, curing the flexible shell with the polymer-coated beads adhered thereto, and causing or allowing the polymer-coated beads to be removed from the shell thereby leaving impressions of the particles in the shell to create an open-pored structure on a surface thereof.

In some embodiments, the flexible shell is formed of a silicone elastomer. For instance, the flexible shell may be formed of a plurality of layers of different silicone elastomers, or the flexible shell may be formed of a single homogeneous layer of a silicone elastomer.

In some embodiments, the step of forming the flexible shell may comprise dipping a mandrel into a liquid dispersion of elastomeric material. Alternatively, the step of forming comprises rotational molding.

In some embodiments, the step of adhering comprises dipping the flexible shell into a liquid containing the polymer-coated beads, for example, a liquid dispersion or emulsion of polymer-coated beads. Prior to the step of dipping the flexible shell, the process may also include applying a tack coat layer onto the flexible shell.

In some embodiments, the solvent is an aqueous composition, for example, water. In some embodiments, the polymer-coated beads comprise a suitable solid material, which is provided in a rounded particulate form, and which is capable of being adhered to a shell, for example, an uncured elastomer shell, and is capable of being dissolved, for example, using a solvent, thereby leaving open, rounded pores in the shell.

In some embodiments, the polymer-coated beads have a substantially uniform particle size of between about 150 μm and about 1450 μm. More specifically, the beads have a maximum particle size range selected from a group of ranges consisting of (1) a range between about 180 μm and about 425 μm, (2) a range between about 425 μm and about 850 μm, and (3) a range between about 850 μm and about 1450 μm. In some embodiments, about 90% of the particles are in the selected particle size range. Size selection can be accomplished by sieving with the desired size sieve(s).

In some embodiments, a soft prosthetic breast implant can be formed by a process comprising the steps of forming a flexible shell of silicone elastomer in the shape of a breast implant, adhering on the exterior of the flexible shell a substantially even distribution of polymer-coated beads, curing the flexible shell with the polymer-coated beads adhered thereto, and exposing the flexible shell to a suitable solvent for a sufficient amount of time to dissolve the polymer-coated beads thereby forming an open-pored structure on the exterior of the flexible shell.

In accordance with some embodiments, an implant formed in accordance with the present process can be far superior to an implant made using conventional porogens or sacrificial materials. For example, in some embodiments, at least one, at least two, or all three of the physical properties of ultimate break force, ultimate elongation, or ultimate tensile strength of an implant formed in accordance with the present process can be superior to an implant made using substantially the same process and the same materials but using a different porogen/sacrificial material than the presently disclosed polymer-coated beads.

The step of forming the flexible shell may comprise dipping a mandrel into a liquid dispersion of a shell material, or rotational molding. In some embodiments, the step of forming the flexible shell comprises forming a shell with an opening and the process further includes attaching a patch to cover the opening. The patch may be an unvulcanized elastomer and is attached prior to the step of curing. Alternatively, the step of forming the flexible shell comprises forming a shell with an opening and the process further includes attaching a valve, for example, a one-way valve to cover the opening. The polymer-coated beads may comprise a sugar, for example sucrose, and polyethylene glycol.

### Example

The following Example is provided for illustrative purposes only, and is not intended to be limiting of the scope of the present disclosure.

Eleven designs of experiment (“DOE”) were conducted for coating rounded sugar particles with the parameters as listed in Table 1.

TABLE 1
Experimental Parameters for polymer-coating process

| Experiment | Temper- | Spray | Nonaqueous Solvent |
|---|---|---|---|
| (DOE) No. | ature (° C.) | Rate (g/min) | Content (wt %) |
| 1 | 25.0 | 65 | 50 |
| 2 | 32.5 | 45 | 75 |
| 3 | 40.0 | 25 | 100 |
| 4 | 32.5 | 45 | 75 |
| 5 | 25.0 | 25 | 100 |
| 6 | 40.0 | 25 | 50 |
| 7 | 25.0 | 65 | 100 |
| 8 | 40.0 | 65 | 100 |
| 9 | 32.5 | 45 | 75 |
| 10 | 25.0 | 25 | 50 |
| 11 | 40.0 | 65 | 50 |

For each experiment, 15 kg of sugar spheres, U.S. mesh size 35-40, were Wurster-coated in a Fluid Bed System Model GPCG-15 (Glatt Air Techniques, Germany), with 2 kg of polyethylene glycol (“PEG”) dissolved in 6 kg of solvent mixture. The solvent (ethanol:water) mixtures were 50:50, 75:25, or 100:0. The spray rates were 25 g/min, 45 g/min, or 65 g/min, and the temperature was set at 25° C., 32.5° C., or 40° C. Coated particles were screened with 18-mesh and 60-mesh to remove oversized and undersized (i.e., fines) materials. To compare the surface morphologies, the PEG-coated sugar spheres from each experiment were analyzed under light microscope.

The coated particles were also analyzed using a Malvern Morphologi® G3 to determine the particles' circular equivalent diameter, convexity, and circularity. The percentage of fines recovered was also assessed.

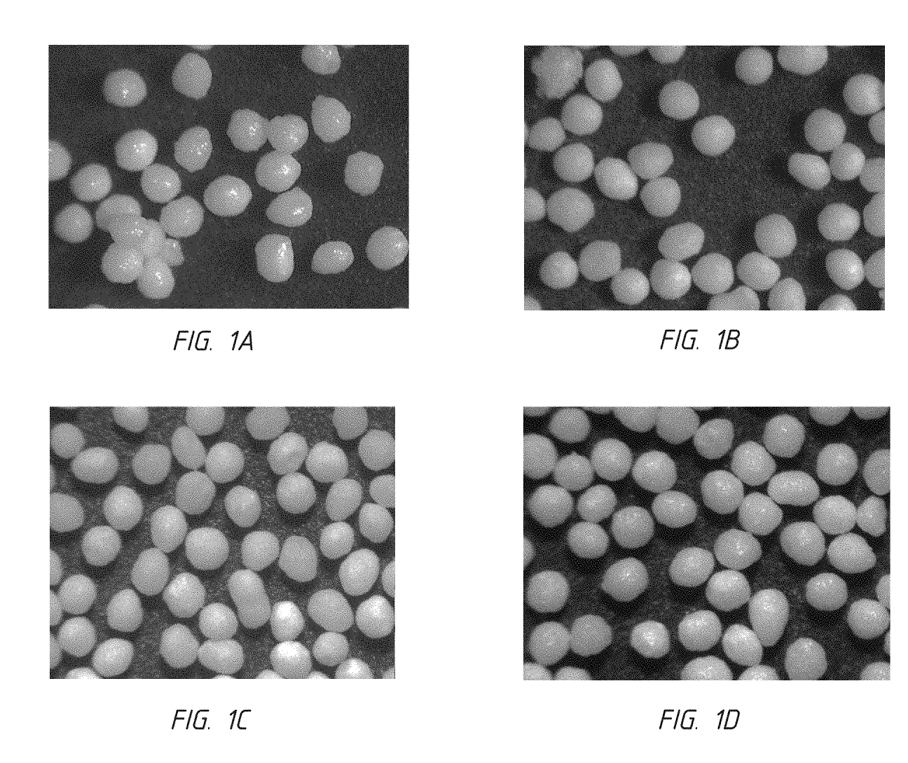

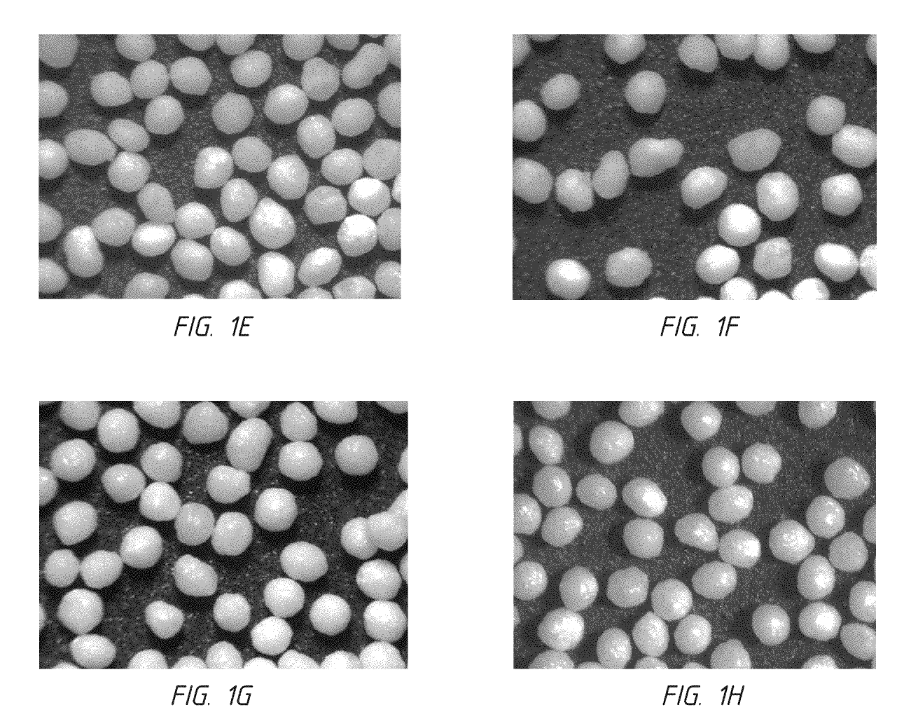

FIGS. 1A-1H provide micrographs of the particles produced by Experiments 1 (FIG. 1A), 3 (FIG. 1B), 5 (FIG. 1C), 6 (FIG. 1D), 7 (1E), 8 (FIG. 1F), 10 (FIG. 1G), and 11 (FIG. 1H). An analysis of these micrographs indicated that the surface morphology of Experiments 3, 5, 7, and 8, which were coated using 100% ethanol solvent, appeared more crystalline compared to the other experiments. In addition, Experiments 5, 7, and 8 had the highest percentages of fines. The micrograph for Experiment 1 (FIG. 1A) showed beads having a bumpy coating. Micrographs of Experiments 10 (FIG. 1G) and 11 (FIG. 1H) showed particles with the smoothest surfaces and higher average convexities and circularities compared to the other 9 batches of coated sugar spheres.

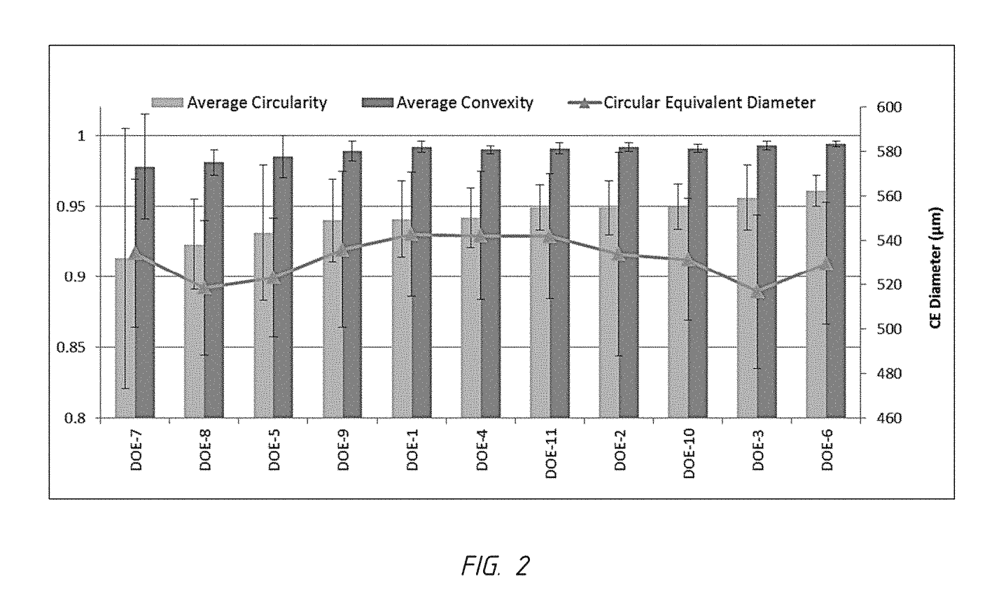

A graph of the circularity and convexity analyses of the polymer-coated beads from Experiments 1-11 is provided in FIG. 2. This graphs shows that Experiments 7, 8, and 5 yielded the least circular beads and lowest average convexity among the 11 experiments. Although Experiment 6 had the highest average circularity, it generated some fines and had a smaller mean diameter. The results of the experiments indicates that coating that yielded the best surface properties was achieved when the coating solutions were prepared with at least 50% ethanol, for example, 50% ethanol.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A process for making polymer-coated beads, the process comprising: providing a particulate material, the particulate material comprising particles; depositing the particulate material into an enclosed zone; fluidizing the particles in the enclosed zone; introducing an aqueous dispersion into the enclosed zone containing the fluidized particles, the aqueous dispersion comprising a polymer component and a solvent component, wherein the solvent component comprises at least 25% by weight of a nonaqueous solvent; allowing the aqueous dispersion to coat the fluidized particles; and allowing or causing the solvent component to evaporate, thereby leaving a polymer coating on the particles, wherein the polymer coating is at least about 10 μm thick.

Clause 2. The process of Clause 1, wherein the particles are spherical.

Clause 3. The process of Clause 1 or Clause 2, wherein the particles are water-soluble.

Clause 4. The process of Clause 3, wherein the particles comprise a salt.

Clause 5. The process of Clause 4, wherein the salt is selected from the group consisting of sodium chloride, potassium chloride, lithium chloride, magnesium chloride, calcium chloride, ammonium chloride, sodium iodide, potassium iodide, lithium iodide, magnesium iodide, calcium iodide, ammonium iodide, sodium bromide, potassium bromide, lithium bromide, magnesium bromide, calcium bromide, ammonium bromide, sodium carbonate, potassium carbonate, lithium carbonate, magnesium carbonate, ammonium carbonate, sodium bicarbonate, potassium bicarbonate, lithium bicarbonate, ammonium bicarbonate, sodium nitrate, potassium nitrate, lithium nitrate, magnesium nitrate, calcium nitrate, ammonium nitrate, sodium acetate, potassium acetate, lithium acetate, magnesium acetate, calcium acetate, ammonium acetate, sodium phosphate, potassium phosphate, lithium phosphate, magnesium phosphate, calcium phosphate, ammonium phosphate, sodium sulfate, potassium sulfate, lithium sulfate, magnesium sulfate, calcium sulfate, ammonium sulfate, and combinations thereof.

Clause 6. The process of Clause 5, wherein the salt comprises sodium chloride.

Clause 7. The process of Clause 3, wherein the particles comprise a sugar.

Clause 8. The process of Clause 7, wherein the sugar is selected from the group consisting of sucrose, fructose, lactose, galactose, mannose, dextrose, glucose, and combinations thereof.

Clause 9. The process of Clause 8, wherein the sugar comprises sucrose.

Clause 10. The process of any one of the preceding Clauses, wherein the polymer coating comprises a polymer selected from the group consisting of poly(alkylene oxide), poly(acrylamide), poly(acrylic acid), poly(acrylamide-co-acrylic acid), poly(acrylamide-co-diallyldimethylammonium chloride), poly(acrylonitrile), poly(allylamine), poly(amide), poly(anhydride), poly(butylene), poly(ε-caprolactone), poly(carbonate), poly(ester), poly(etheretherketone), poly(ethersulphone), poly(ethylene), poly(ethylene alcohol), poly(ethylenimine), polyethylene glycol, poly(ethylene oxide), poly(glycolide) ((like poly(glycolic acid)), poly(hydroxy butyrate), poly(hydroxyethylmethacrylate), poly(hydroxypropylmethacrylate), poly(hydroxystyrene), poly(imide), poly(lactide), poly(L-lactic acid), poly(D,L-lactic acid), poly(lactide-co-glycolide), poly(lysine), poly(methacrylate), poly(methacrylic acid), poly(methylmethacrylate), poly(orthoester), poly(phenylene oxide), poly(phosphazene), poly(phosphoester), poly(propylene fumarate), poly(propylene), poly(propylene glycol), poly(propylene oxide), poly(styrene), poly(sulfone), poly(tetrafluoroethylene), poly(vinyl acetate), poly(vinyl alcohol), poly(vinyl chloride), poly(vinylidene fluoride), poly(vinyl pyrrolidone), poly(urethane), collagen, gelatin, any copolymer thereof (like poly(ethylene oxide) poly(propylene oxide) copolymers (poloxamers), poly(vinyl alcohol-co-ethylene), poly(styrene-co-allyl alcohol, and poly(ethylene)-block-poly(ethylene glycol), and/or any mixtures thereof.

Clause 11. The process of Clause 10, wherein the polymer coating comprises polyethylene glycol.

Clause 12. The process of any one of the preceding Clauses, wherein the step of introducing an aqueous dispersion into the enclosed zone comprises spraying, injecting, misting, nebulizing, or aerosolizing the aqueous dispersion.

Clause 13. The process of Clause 12, wherein the step of introducing an aqueous dispersion comprises spraying the aqueous dispersion into the enclosed zone.

Clause 14. The process of Clause 13, wherein the aqueous dispersion is sprayed into the enclosed zone as a top spray, a tangential spray, a bottom spray, or combinations thereof.

Clause 15. The process of Clause 14, wherein the aqueous dispersion is sprayed into the enclosed zone as a bottom spray.

Clause 16. The process of any one of Clauses 13 to 15, wherein the aqueous dispersion is sprayed into the enclosed zone at a rate of between about 25 grams/min and about 65 grams/min.

Clause 17. The process of any one of Clauses 13 to 16, wherein the temperature in the enclosed zone during the spraying is between about 25° C. and about 40° C.

Clause 18. The process of any one of the preceding Clauses, wherein the nonaqueous solvent comprises a solvent selected from a C1 to C4 alcohol, acetone, methyl ethylene ketone, dimethylformamide, ethylene glycol, dichloromethane, chloroform, and combinations thereof.

Clause 19. The process of Clause 17, wherein the nonaqueous solvent comprises a solvent selected from ethanol, methanol, and combinations thereof.

Clause 20. The process of Clause 19, wherein the nonaqueous solvent comprises ethanol.

Clause 21. The process of any one of the preceding Clauses, wherein the solvent component comprises between about 25% and about 90% by weight of a nonaqueous solvent.

Clause 22. The process of Clause 21, wherein the solvent component comprises between about 30% and about 80% by weight of a nonaqueous solvent.

Clause 23. The process of Clause 21, wherein the solvent component comprises between about 40% and about 60% by weight of a nonaqueous solvent.

Clause 24. The process of Clause 21, wherein the solvent component comprises between about 50% and about 60% by weight of a nonaqueous solvent.

Clause 25. The process of Clause 21, wherein the solvent component comprises between about 45% and about 55% by weight of a nonaqueous solvent.

Clause 26. The process of Clause 21, wherein the solvent component comprises between about 60% and about 90% by weight of a nonaqueous solvent.

Clause 27. The process of Clause 21, wherein the solvent component comprises between about 60% and about 80% by weight of a nonaqueous solvent.

Clause 28. The process of Clause 21, wherein the solvent component comprises between about 70% and about 80% by weight of a nonaqueous solvent.

Clause 29. The process of Clause 21, wherein the solvent component is about 50% by weight ethanol and 50% by weight water.

Clause 30. The process of Clause 21, wherein the solvent component is about 75% by weight ethanol and 25% by weight water.

Clause 31. The process of any one of the preceding Clauses, wherein the polymer-coated beads have a mean circularity of at least about 0.6.

Clause 32. The process of any one of the preceding Clauses, wherein the polymer-coated beads have a mean circularity of at least about 0.7.

Clause 33. The process of any one of the preceding Clauses, wherein the polymer-coated beads have a mean circularity of at least about 0.80.

Clause 34. The process of any one of the preceding Clauses, wherein the polymer-coated beads have a mean circularity of at least about 0.90.

Clause 35. The process of any one of the preceding Clauses, wherein the polymer-coated beads have a mean circularity of at least about 0.95.

Clause 36. The process of any one of the preceding Clauses, wherein the polymer-coated beads have a diameter of about 100 μm to about 1,000 μm.

Clause 37. The process of any one of the preceding Clauses, wherein the polymer-coated beads have a diameter of about 200 μm to about 900 μm.

Clause 38. The process of any one of the preceding Clauses, wherein the polymer-coated beads have a diameter of about 300 μm to about 800 μm.

Clause 39. The process of any one of the preceding Clauses, wherein the polymer-coated beads have a diameter of about 400 μm to about 700 μm.

Clause 40. The process of any one of the preceding Clauses, wherein the polymer-coated beads have a diameter of about 500 μm to about 600 μm.

Clause 41. The process of any one of the preceding Clauses, wherein the polymer coating is at least about 15 μm thick.

Clause 42. The process of any one of the preceding Clauses, wherein the polymer coating is at least about 20 μm thick.

Clause 43. The process of any one of the preceding Clauses, wherein the polymer coating is at least about 25 μm thick.

Clause 44. The process of any one of the preceding Clauses, wherein the polymer coating is at least about 30 μm thick.

Clause 45. The process of any one of the preceding Clauses, wherein the aqueous dispersion comprises about 1% by weight to about 40% by weight of the polymer component.

Clause 46. The process of any one of the preceding Clauses, wherein the aqueous dispersion comprises about 20% by weight to about 30% by weight of the polymer component.

Clause 47. The process of any one of the preceding Clauses, wherein the aqueous dispersion comprises about 25% by weight of the polymer component.

Clause 48. The process of any one of the preceding Clauses, wherein the process has a duration of about 1 hour to about 10 hours.

Clause 49. The process of any one of the preceding Clauses, wherein the process has a duration of at least about 4 hours.

Clause 50. The process of any one of the preceding Clauses, wherein the process has a duration of at least about 6 hours.

Clause 51. The process of any one of the preceding Clauses, wherein the enclosed zone comprises part of an apparatus for fluidized bed coating.

Clause 52. A polymer-coated bead produced by the process of any one of the preceding Clauses.

Clause 53. A process for manufacturing a soft prosthetic breast implant, the process comprising: forming a flexible shell of silicone elastomer, the silicone elastomer having a thickness; adhering on an exterior of the flexible shell an even distribution of the polymer-coated beads of Clause 52; curing the flexible shell with the polymer-coated beads adhered thereto; removing the polymer-coated beads thereby forming an open-pored structure on the exterior of the flexible shell, such that the exterior of the flexible shell exhibits an undulating topography, the open-pored structure comprising round cavities defined by impressions of the polymer-coated beads; and processing the flexible shell, such that it forms a closed envelope; wherein the open-pored structure does not extend through an entire thickness of the silicone elastomer.

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

In one or more aspects, the terms “about,” “substantially,” and “approximately” may provide an industry-accepted tolerance for their corresponding terms and/or relativity between items, such as from less than one percent to five percent.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A process for making polymer-coated beads, the process comprising:
providing a particulate material, the particulate material comprising particles;
depositing the particulate material into an enclosed zone;
fluidizing the particles in the enclosed zone;
introducing an aqueous dispersion into the enclosed zone containing the fluidized particles, the aqueous dispersion comprising a polymer component and a solvent component, wherein the solvent component comprises at least 25% by weight of a nonaqueous solvent;
allowing the aqueous dispersion to coat the fluidized particles; and
allowing or causing the solvent component to evaporate, thereby leaving a polymer coating on the particles, wherein the polymer coating is at least about 10 μm thick.

2. The process of claim 1, wherein the particles are spherical.

3. The process of claim 2, wherein the particles are water-soluble.

4. The process of claim 3, wherein the particles comprise a salt.

5. The process of claim 4, wherein the salt comprises sodium chloride.

6. The process of claim 3, wherein the particles comprise a sugar.

7. The process of claim 6, wherein the sugar comprises sucrose.

8. The process of claim 1, wherein the polymer coating comprises polyethylene glycol.

9. The process of claim 1, wherein the step of introducing an aqueous dispersion comprises spraying the aqueous dispersion into the enclosed zone.

10. The process of claim 9, wherein the aqueous dispersion is sprayed into the enclosed zone as a bottom spray.

11. The process of claim 9, wherein the aqueous dispersion is sprayed into the enclosed zone at a rate of between about 25 grams/min and about 65 grams/min.

12. The process of claim 9, wherein the temperature in the enclosed zone during the spraying is between about 25° C. and about 40° C.

13. The process of claim 1, wherein the nonaqueous solvent comprises a solvent selected from ethanol, methanol, and combinations thereof.

14. The process of claim 13, wherein the nonaqueous solvent comprises ethanol.

15. The process of claim 1, wherein the solvent component comprises between about 30% and about 80% by weight of a nonaqueous solvent.

16. The process of claim 1, wherein the solvent component is about 50% by weight ethanol and 50% by weight water.

17. The process of claim 1, wherein the solvent component is about 75% by weight ethanol and 25% by weight water.

18. The process of claim 1, wherein the polymer-coated beads have a mean circularity of at least about 0.95.

19. The process of claim 1, wherein the polymer-coated beads have a diameter of about 100 μm to about 1,000 μm.

20. The process of claim 1, wherein the polymer-coated beads have a diameter of about 500 μm to about 600 μm.
